(19) United States
(12) Patent Application Publication
(10) Pub. No.: US 2022/0008406 A1
(43) Pub. Date: Jan. 13, 2022

(54) USE OF NUCIFERIN AND LOTUS LEAF EXTRACT IN THE PREPARATION OF A MEDICAMENT FOR TREATING ATROPHIC GASTRITIS AND/OR BLOCKING TRANSFORMATION OF GASTRITIS INTO CANCER

(71) Applicants: Shao LI, Beijing (CN); TSINGHUA UNIVERSITY, Beijing (CN)

(72) Inventor: SHAO LI, BEIJING (CN)

(21) Appl. No.: 17/276,146

(22) PCT Filed: Sep. 9, 2019
(86) PCT No.: PCT/CN2019/104997

§ 371 Date: Mar. 13, 2021

(30) Foreign Application Priority Data
Sep. 13, 2018 (CN) 201811065059.3
Jun. 18, 2019 (CN) 201910530555.X

(51) Int. Cl. A61K 31/473; A61K 36/48; A61P 1/04; A23L 33/00; A23L 33/105

(52) U.S. Cl. CPC A61K 31/473; A61K 36/48; A23V 2002/00; A23L 33/40; A23L 33/105; A61P 1/04

(57) ABSTRACT

The present invention provides a use of a substance in the preparation of a product for treating chronic atrophic gastritis, protecting gastric mucosa, treating gastric precancerous lesions, blocking transformation of gastritis into cancer, and preventing occurrence of gastric cancer, the product being one selected from a medicament, a healthcare product and a foodstuff, and the substance being selected from the group consisting of: nuciferin, a nuciferin derivative, a lotus leaf extract, and a mixture of at least two selected from the above substances. The use includes at least one of treating chronic atrophic gastritis, protecting gastric mucosa, blocking transformation of gastritis into cancer, treating gastric precancerous lesions, and preventing occurrence of gastric cancer. In vitro experiments showed that nuciferin can block transformation of gastritis into cancer and cell proliferation. In vivo experiments showed that nuciferin and lotus leaf extract can treat atrophic gastritis in rats (especially chronic atrophic gastritis with intestinal metaplasia or dysplasia), protect rats from gastric mucosal damage and inhibit abnormal proliferation of gastrointestinal stem cells in Drosophila. Clinical tests indicated that lotus leaf extract can improve symptoms and objective indicators in patients with atrophic gastritis or gastric precancerous lesions.

### TECHNICAL FIELD

The present invention belongs to the field of natural small molecule compounds of traditional Chinese medicine and traditional Chinese medicine extracts, and specifically relates to uses of nuciferine and nuciferine extracts in preparation of medicines, health care products and foods for treating chronic atrophic gastritis, treating gastric precancerous lesions and/or blocking the occurrence of gastritis-cancer transformation.

### BACKGROUND ART

Chronic inflammation is closely related to tumorigenesis [1]. Current epidemiological investigations and multiple scientific research results show that in addition to genetic factors, most tumors are also closely related to environmental factors and lifestyles, such as smoking, diet, and infections. These carcinogenic factors can lead to different forms of cancer, and uncontrollable inflammation can promote tumorigenesis [2,3]. Especially and typically, the occurrence of gastric cancer is closely related to gastritis and Helicobacter pylori infection [4,5]. According to statistics from the National Cancer Registry, more than half of all cancer-related deaths in China were cancers of the digestive system. Among them, the incidence and mortality of gastric cancer are among the highest, and almost half of the global gastric cancers occur in China. Gastric cancer is the number one tumor in both incidence and mortality in rural areas in China [6,7]. Early prevention and control of gastric cancer is very important. The lack of effective prevention and treatment measures for gastritis cancer transformation is an important reason for the high incidence of gastric cancer.

In an inflammatory environment, immune cells can release a variety of cytokines and chemokines. By activating key transcription factors such as NF-κB, STAT, and SMAD in somatic cells, they can cause changes in somatic genome and epigenetics, leading to abnormal cell proliferation, apoptosis and differentiation. At the same time, there are a large number of active oxygen ROS, active nitrogen and other groups in the inflammatory microenvironment, which not only affect the epigenome changes of somatic cells by influencing the chemical modification of DNA and proteins, but also play a key role in DNA damage, ultimately causing body cellular mutation and tumorigenesis [8-10]. How to treat mild inflammation to severe inflammation and block the transformation of inflammatory cancer is extremely important to prevent tumorigenesis. In recent years, the discovery of drugs that can treat chronic inflammation and block or inhibit the transformation of inflammation to cancer has also become a focus of attention.

Preventing tumors and treating tumors are two completely different concepts. Although there are many anti-tumor drugs at present, drugs that can effectively inhibit the transformation of inflammation-cancer and prevent tumorigenesis have so far been rare. Studies have reported that the incidence of colon cancer in people who take aspirin for a long time is reduced by 24% [11]. In addition, celecoxib, a COX-2 inhibitor clinically used to treat arthritis (trade name Celebrex, English trade name Celebrex, the world's first selective cyclooxygenase-2 inhibitor on the market) is also used to prevent bowel cancer caused by enteritis. However, different parts of the digestive system (such as the esophagus, stomach, intestine, liver, pancreas, etc.) have different mechanisms of inflammation-cancer transformation. Aspirin and celecoxib are aimed at the inflammation-cancer transformation of enteritis cancer, not the inflammation-cancer transformation of gastritis and cancer. And these two drugs all have certain side effects. In short, there is still a lack of drugs that can effectively prevent gastritis and cancer transformation.

The lotus leaf is the leaf of Nelumbo nuficera Gaertn, which are cultivated in the north and south of China. Lotus leaf has been included in the list of Chinese medicines that are both food and medicine in the No. 45 document issued by the Ministry of Health of the People's Republic of China (1991) in November 1991. Lotus leaf was also recently listed by the National Health and Family Planning Commission in 2018 in its “Medicine and Food Homologous” catalog. Nuciferine, a kind of apophine-type alkaloid compound, is one of the main active ingredients of Chinese medicine lotus leaf. Nuciferine can be dissolved in organic solvents such as methanol and chloroform, and its extraction generally uses sun-dried and crushed lotus leaves as raw materials, using organic solvent methods. In order to improve the yield, ultrasonic, critical extraction and supercritical extraction are generally assisted. Nuciferine can improve hyperlipidemia [12] and reduce lipase activity [13]. Many studies have shown that nuciferine has pharmacological activity in treating various diseases, such as cardiovascular disease and cancer. It is currently reported that nuciferine has anti-atherosclerosis, antibacterial, anti-viral and anti-tumor biological activities [14-16]. Lotus leaf extract refers to the material obtained from lotus leaf as a raw material and extracted with water or organic solvents. The main components include lotus leaf alkaloids, lotus leaf flavonoids, citric acid, oxalic acid, etc. Lotus leaf extract has been reported to have biological activities such as lowering blood fat, weight loss, and scavenging free radicals [17,18]. As the current weight loss health products, lotus leaf extract and lotus leaf extract have low price and good safety, which provide a great possibility for their clinical use in the treatment of chronic atrophic gastritis and blocking gastritis cancer transformation. The inventor's previous research found for the first time that nuciferine has an inhibitory effect on the transformation of enteritis to bowel cancer, and obtained the invention patent [19]. But enteritis to bowel cancer transformation and gastritis to stomach cancer transformation are two distinct disease processes. Literature research has not found any report on the therapeutic effects of lotus leaf extract on gastric cancer caused by gastritis, gastric precancerous lesions, or gastritis-gastric cancer transformation.

### SUMMARY OF THE INVENTION

Drugs for tumor prevention that block inflammation-cancer transformation is a specialized field independent of anti-tumor drugs. Since the research of anti-tumor drugs is directly conducted with tumor cell lines or tumor tissues, clinically, it corresponds to the treatment of middle and advanced tumors. This is a completely different concept from preventing tumors, especially blocking the transformation of inflammatory cells or tissues into tumors.

The purpose of the present invention is to provide use of nuciferine and lotus leaf extract to treat precancerous lesions of chronic atrophic gastritis accompanied by intestinal metaplasia or dysplasia and to block the occurrence of gastritis-gastric cancer transformation, which use can result in the prevention of gastric cancer. The traditional Chinese medicine of the medicine and food homology involved in the invention is nuciferine and lotus leaf extract, the extraction technology of which is mature and of high extraction rate and low cost. Nuciferine and lotus leaf extract are mainly extracted from the natural plant lotus leaf.

Through a series of experiments, the inventors confirmed that nuciferine and lotus leaf extract have the effect of treating chronic atrophic gastritis and gastric precancerous lesions, and preventing the transformation of gastritis to gastric cancer.

The present invention provides the use of a substance in preparing a product for preventing the occurrence of gastric cancer, wherein said product is one selected from the group consisting of medicines, health care products, and foods, and the substance is selected from nuciferine, derivatives of nuciferine, lotus leaf extracts, and a mixture of the same. The prevention of gastric cancer includes at least one of treating chronic atrophic gastritis, protecting gastric mucosa, blocking gastritis to gastric cancer transformation, preventing gastritis from turning into gastric cancer, treating gastric precancerous lesions, and preventing gastric cancer from occurring.

According to one aspect of the present invention, there is provided a use of a substance for preparing a product to treat chronic atrophic gastritis, gastric precancerous lesions and to block gastritis to gastric cancer transformation, where the substance is one selected from:

nuciferine,

nuciferine derivatives,

lotus leaf extracts,

a mixture containing at least two selected from the group consisting of nuciferine, nuciferine derivatives, and lotus leaf extracts.

According to a further aspect of the present invention, the above-mentioned nuciferine belongs to the apophine-type alkaloid compound, its molecular formula is C19H21NO2, and its molecular weight is 295.38 g/mol; its structural formula is:

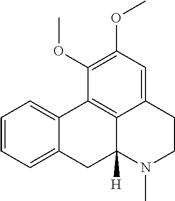 [SMILES: COc1cc2c3c(c1OC)-c1ccccc1C[C@H]3N(C)CC2]

According to another aspect of the present invention, there is provided a use of a substance for preparing a product for prevention of tumor occurrence, wherein said product is one selected from the group consisting of medicines, health care products, and foods, and said substance is one selected from:

nuciferine,

nuciferine derivatives,

lotus leaf extracts,

a mixture containing at least two selected from the group consisting of nuciferine, nuciferine derivatives, and lotus leaf extracts.

According to a further aspect of the present invention, the prevention of tumor occurrence includes at least one of treating chronic atrophic gastritis, protecting gastric mucosa, blocking gastritis to gastric cancer transformation, treating gastric precancerous lesions, and preventing gastric cancer occurrence.

According to another aspect of the present invention, there is provided a product, which contains as its active ingredient one selected from the following substances:

nuciferine,

nuciferine derivatives,

lotus leaf extracts,

a mixture containing at least two selected from the group consisting of nuciferine, nuciferine derivatives, and lotus leaf extracts,

and which contains other auxiliary materials,

wherein said product is one selected from medicines, health care products, and foods.

According to a further aspect of the present invention, the above-mentioned medicines have a dosage form selected from tablets, capsules, pills, injections, sustained release agents, controlled release agents, powders, beverages and the like.

According to a further aspect of the present invention, the above-mentioned health care products have a dosage form selected from tablets, capsules, pills, injections, sustained-release agents, controlled-release agents, powders, beverages and the like.

### DESCRIPTION OF THE DRAWINGS

FIG. 1 shows the statistical results of the chronic inflammation pathological state of the stomach tissue in the experiment of nuciferine treatment of chronic gastritis with atrophy with intestinal metaplasia in an embodiment of the present invention.

FIGS. 2A-2E show HE staining diagrams of gastric tissue pathological sections under normal, chronic gastritis atrophy with intestinal metaplasia, and nuciferine intervention in representative individuals of the corresponding group in an embodiment of the present invention.

FIG. 3 shows the statistical results of the chronic inflammation pathological state of the stomach tissue in the experiment of lotus leaf extract treatment of chronic gastritis with atrophy with intestinal metaplasia in an embodiment of the present invention.

FIGS. 4A-4D show HE staining diagrams of gastric tissue pathological sections under normal, chronic gastritis atrophy with intestinal metaplasia, and lotus leaf extract intervention in representative individuals of the corresponding group in an embodiment of the present invention.

FIGS. 5A to 5E show the improvement of gastric mucosal damage of representative individuals in the corresponding groups of normal, gastric mucosal injury models, nuciferine intervention and lotus leaf extract intervention in an embodiment of the present invention.

FIG. 6 shows the statistical effect of nuciferine of inhibiting the abnormal proliferation of Drosophila gastrointestinal stem cells and mucosal ulceration in an embodiment of the present invention.

FIG. 7 shows the statistical effect of lotus leaf extract of inhibiting the abnormal proliferation of Drosophila gastrointestinal stem cells and mucosal ulceration in an embodiment of the present invention.

FIGS. 8A-8G show fluorescence staining diagrams of the inhibition of abnormal proliferation of gastrointestinal stem cells and improvement of mucosal ulceration of representative individuals in the model group, the nuciferine intervention group and the lotus leaf extract intervention group in an embodiment of the present invention.

FIG. 9 shows the statistical results of the inhibitory effect of nuciferine on the growth of gastritis to gastric cancer transformed cells in an embodiment of the present invention.

FIG. 10 shows the result that the half inhibitory concentration (IC50) of the growth inhibitory effect of nuciferine on the growth of cells transformed from gastritis to gastric cancer is 92.72 μmol/L in an embodiment of the present invention.

FIG. 11 shows the improvement of lotus leaf extract on the conditions of patients with chronic atrophic gastritis or gastric precancerous lesions in an embodiment of the present invention.

### DETAILED DESCRIPTION

According to one aspect of the present invention, there is provided a novel medicine for treating atrophic gastritis or gastric precancerous lesions and for blocking transformation of gastritis to gastric cancer, wherein the active substances contained in the medicine include nuciferine and/or lotus leaf extract. Lotus leaf extract is derived from the plant lotus leaf and is obtained by water or organic solvent extraction. The inventors have confirmed through experiments that nuciferine and/or lotus leaf extract are safe and can effectively treat chronic atrophic gastritis, protect the gastric mucosa, block the transformation of gastritis to gastric cancer, prevent the transformation of gastritis into gastric cancer, treat precancerous lesions and prevent stomach cancer occurrence.

According to another aspect of the present invention, there is provided the use of a plant or natural product containing nuciferine in preparing a medicine for treating chronic atrophic gastritis, inhibiting the transformation of gastritis to gastric cancer and/or preventing the occurrence of gastric cancer.

The nuciferine (English name nuciferine) used to realize the above aspects of the present invention belongs to apophine-type alkaloids, has the molecular formula C19H21NO2, has a molecular weight of 295.38 g/mol; and has the structural formula as:

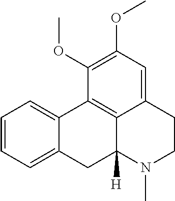 [SMILES: COc1cc2c3c(c1OC)-c1ccccc1C[C@H]3N(C)CC2]

In the uses according to the present invention, the prevention of tumor occurrence includes at least one of treating chronic atrophic gastritis, protecting gastric mucosa, blocking transformation of gastritis to gastric cancer, treating gastric precancerous lesions, and preventing tumor occurrence.

In the process of carrying out and achieving the present invention, the inventors conducted a series of experiments. In the following description of the embodiments, the results of these experiments are given. Results of these experimental show that nuciferine and lotus leaf extract can, at a safe dose, significantly treat atrophic gastritis (especially chronic atrophic gastritis with intestinal metaplasia or dysplasia) and/or block transformation of gastritis to gastric cancer. The inventor's findings in the above experiments and in other work related to the present invention include:

(1) Nuciferine could Treat Chronic Atrophic Gastritis with Intestinal Metaplasia

An animal model of chronic atrophic gastritis induced by sodium deoxycholate and ammonia water was selected [20]. After the model was successfully established, celecoxib and nuciferine were given by gavage. The pathological results showed that nuciferine effectively treated rats with chronic atrophic gastritis, reversed intestinal metaplasia, returned the stomach of some of the rats to a normal state, and reduced the inflammation in most of the rats to a state of mild chronic non-atrophic gastritis.

(2) Lotus Leaf Extract Treated Chronic Atrophic Gastritis with Dysplasia

The rat model of chronic atrophic gastritis was induced by the combination of sodium deoxycholate, ethanol and ammonia water [21], and the lotus leaf extract was given to the stomach after successful modeling. The pathological results showed that the lotus leaf extract effectively treated rats with chronic atrophic gastritis with dysplasia, returned the stomach of some rats to a normal state and relieved the gastritis of some rats.

(3) Nuciferine and Lotus Leaf Extract Protected Gastric Mucosa from Damage

After continuous administration of lotus leaf extract and lotus leaf extract in rats, gastric mucosal damage was induced by absolute ethanol [22], and the gastric mucosal damage was observed under a microscope after dissection. The rats in the model group were seen to have different degrees of gastric mucosal damage, and the rats in the nuciferine group and the lotus leaf extract group were seen to have significantly improved gastric mucosal damage compared with the model group.

### (4) Nuciferine and Lotus Leaf Extract Inhibited Abnormal Proliferation of Gastric Stem Cells

The gastrointestinal tumorigenesis model reported in the publication “Cell Stem Cell” of “Cell” Press [23] was select. Dextran sulfate sodium (DSS) was used to induce abnormal proliferation of Drosophila gastrointestinal stem cells to simulate the process of tumorigenesis. During the modeling period, nuciferine and lotus leaf extract were given. The results under the microscope showed that both nuciferine and lotus leaf extract effectively inhibited the abnormal proliferation of Drosophila gastrointestinal stem cells and mucosal ulceration.

### (5) Nuciferine Inhibited Cell Proliferation Related to Transformation of Gastritis to Gastric Cancer

The cell model of transformation of gastritis to gastric cancer reported by the well-known journals in tumor research and the British Natural Press “Oncogene” (Oncogene) was selected, with chemical mutagen N-methyl N-nitronitrosoguanidine (N-methyl-N′-nitro-N-nitrosoguanidine, MNNG) stimulated human gastric epithelial cells GES-1 model of transformation of gastritis to gastric cancer as the object [24], by MTT method (3-(4,5-dimethylthiazole-2)-2, 5-Diphenyltetrazolium bromide colorimetric method), the median inhibitory concentration (IC50) of nuciferine on GES-1 model cells of transformation of gastritis to gastric cancer stimulated by MNNG was measured at 92.72 μmol/L. The results show that nuciferine can inhibit the proliferation of cells related to transformation of gastritis to gastric cancer and block the process of transformation of gastritis to gastric cancer.

(6) Lotus Leaf Extract Improved the Symptoms and Indicators of Patients with Atrophic Gastritis with intestinal metaplasia or dysplasia

According to the new Sydney standard, patients (aged 18-70 years old) who were diagnosed as chronic atrophic gastritis with intestinal metaplasia or dysplasia by endoscopic pathology were included, and the subjects were treated with lotus leaf extract after completing the informed consent form in a clinical observation test of transformation of gastritis to gastric cancer, and symptoms and laboratory blood indicators related to gastritis (serum gastrin 17) were recorded. The results show that lotus leaf extract can improve the symptoms and objective indicators of patients with chronic atrophic gastritis with intestinal metaplasia or dysplasia.

Hereinafter, the present invention will be explained with reference to specific embodiments. Those skilled in the art can understand that these embodiments are only used to illustrate the present invention, without limiting the scope of the present invention in any way.

### Embodiment 1: Nuciferine has the Effect of Treating Chronic Atrophic Rats with Intestinal Gastritis

The experiment: The experimental animals were from Beijing Weitong Lihua Laboratory Animal Technology Co., Ltd. Thirty SD rats with a specific pathogen free (SPF) grade of 200±20 g were selected, with half males and half males, and randomly divided into 5 groups: normal control group, chronic atrophic gastritis (CAG) model group, the positive drug celecoxib intervention group and the nuciferine intervention group, with 6 rats in each group. The nuciferine was purchased from Chengdu Refines Biotechnology Co., Ltd., with a specification of 5 g and a purity of over 98%. Celecoxib was purchased from Beijing Bailingwei Technology Co., Ltd., with brand J&K, a specification of 1 g, and purity above 98%. Sodium deoxycholate was purchased from Amresco, USA, with a specification of 100 g. The rats in the model group, celecoxib intervention group and nuciferine intervention group were given sodium deoxycholate solution and ammonia to induce chronic atrophic gastritis. 20 mmol/L sodium deoxycholate was administered daily at a dose of 6 ml/kg, twice a week on an empty stomach, 0.1% ammonia water was drunk freely, and the model was continuously modeled for 8 weeks. The modeled rat gastric mucosa showed varying degrees of atrophy accompanied by intestinal metaplasia. After modeling, the celecoxib group and the nuciferine intervention group were given celecoxib 10 mg/kg, nuciferine 40 mg/kg, and 10 mg/kg by gavage administration, once a day. During the experiment, the rats' body weight and food intake were observed.

After 4 weeks of therapeutic administration, the rats gained weight, their food intake was restored compared to the model-building stage, and their coat color returned to normal appearance. At the end of the dosing experiment, after the rats were sacrificed by dislocation, the stomach tissue was quickly taken out and prepared for pathological sectioning. The obtained gastric tissue was cut along the greater curvature of the stomach, washed with saline, blotted dry with filter paper, and fixed with formalin solution (10% formaldehyde solution). The sections were embedded, dehydrated by alcohol, and then made transparent with xylene. And then the sections were waxed and embed in paraffin. In the staining stage, the sections were soaked in xylene solution for 10 minutes each time for 3 times; then they were soaked in absolute ethanol and rinsed in tap water. Finally, the sections were stained with hematoxylin (to stain the nucleus) and eosin (to stain the cytoplasm), and the sections were mounted with neutral gum. Finally, the pathological changes of rat stomach tissue were observed under light microscope.

TABLE 1
Nuciferine treatment of experimental groups
with chronic atrophic gastritis

| | | Number | | |
|---|---|---|---|---|
| | | in the | Total | |
| Group | Dosing content | group | number | label |
| Normal control | Normal saline of | 6 | 30 | A1-A6 |
| group | the same volume | | | |
| CAG model group | Normal saline of | 6 | | B1-B6 |
| | the same volume | | | |
| Positive drug | Celecoxib, dose | 6 | | C1-C6 |
| control group | 10 mg/kg | | | |
| Nuciferine low- | Dose 10 mg/kg | 6 | | D1-D6 |
| dose group | | | | |
| High-dose | Dose 40 mg/kg | 6 | | E1-E6 |
| nuciferine group | | | | |

The Experimental results:

1. Observation of the Phenotype of Rats in the Chronic Atrophic Gastritis Group

The rats in the chronic atrophic gastritis model group showed signs of weight loss, dull hair, lethargy, and loss of appetite. Two rats were taken from the normal group and the model group respectively, and it was found that the rats in the model group showed varying degrees of chronic atrophy with accompanying intestinal metaplasia.

In the experiment, the weight of rats in the model group was significantly lower than that in the normal group. The administration of celecoxib and nuciferine increased the weight of rats, restored appetite and increased activity, indicating that celecoxib and nuciferine can improve the appearance of rats with chronic atrophic gastritis and has a certain therapeutic effect on chronic atrophic gastritis.

2. Observe the Pathological Section of Stomach Tissue Under Light Microscope

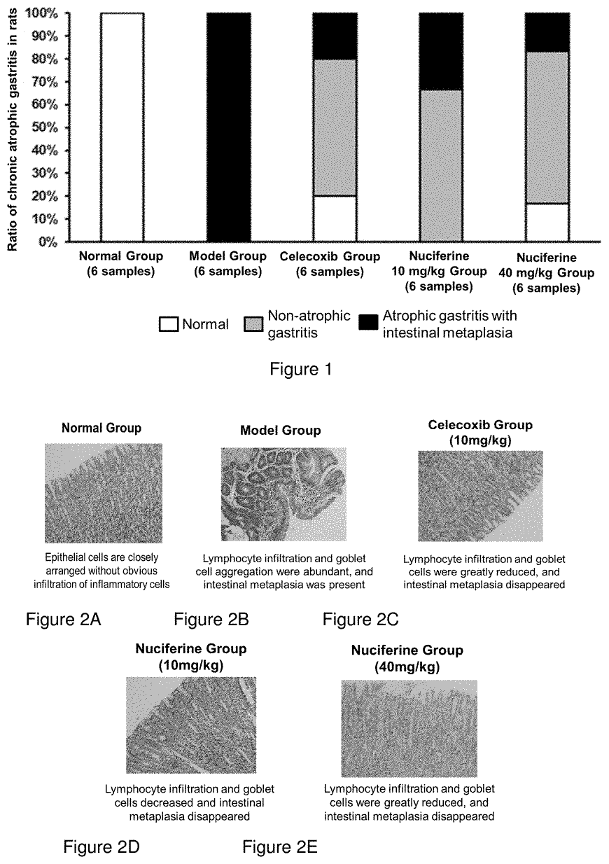

According to the interpretation results of pathological slices, compared with rats in the normal group, the rats in the modeling stage developed chronic atrophic gastritis and mild intestinal metaplasia, in the celecoxib intervention group and the nuciferine intervention group the chronic atrophic gastritis and intestinal metaplasia of rats were effectively treated; the group of celecoxib and the group of high-dose nuciferine had the best effect, with some rats whose stomach returned to normal state, and most of the rest were reduced to mild non-atrophic gastritis. The pathological interpretation results are statistically mapped, and the obtained results are shown in FIG. 1.

Observed under light microscope, the gastric mucosa surface of the rats in the normal group was smooth, the structure was complete, the epithelial cells were tightly arranged, and there was no obvious inflammatory cell infiltration and erosion ulcer formation, as shown in FIG. 2A. In the chronic atrophic gastritis model group, lymphocytes, plasma cells, eosinophils infiltrated and a large number of goblet cells were seen in the lamina propria of the gastric mucosa, most of the inflammatory cells were aggregated, and intestinal metaplasia and local focal area hyperplasia was seen, as shown in FIG. 2B. Compared with the model group, the pathological state of chronic atrophic gastritis in the celecoxib group (FIG. 2C) and the high-dose nuciferine group (FIG. 2E) was significantly reduced, and there were rats that returned to normal. A few lymphocytes, plasma cells and eosinophils were infiltrated in the lamina propria of most rats, and intestinal metaplasia disappeared. In the low-dose nuciferine group (see FIG. 2D), there was a small amount of lymphocytes and plasma cells infiltrated in the lamina propria of the gastric tissue, and the pathological state was reduced to the state of chronic non-atrophic gastritis without ulcer formation.

### Embodiment 2: Lotus Leaf Extract has the Effect of Treating Rats with Chronic Atrophy and Dysplasia Gastritis

The experiment: The experimental animals were from Beijing Sibefu Biotechnology Co., Ltd. Twenty-four SD rats with a specific pathogen free (SPF) 200±20 g (SPF) of 200±20 g were selected, with half male and female, and randomly divided into 4 groups: normal control group, chronic atrophic gastritis (CAG) model group, and two intervention groups of leaf extract, with 6 rats in each group, as shown in Table 2. The lotus leaf extract was purchased from Hebei Chenguang Biotechnology Group Handan Co., Ltd., with a specification of 1 kg, and the content of nuciferine is more than 0.4%. Sodium deoxycholate was purchased from Amresco, USA, with a specification of 100 g. The rats in the model group and lotus leaf extract intervention group were given sodium deoxycholate solution, ammonia and ethanol to induce chronic atrophic gastritis. Gavage of 20 mmol/L sodium deoxycholate and 60% ethanol was made daily at a dose of 6 ml/kg. Gavage on an empty stomach was made twice a week, with free drink of 0.1% ammonia water. After 12 weeks of continuous modeling, the gastric mucosa of the modeled rats showed atrophy accompanied by varying degrees of intestinal metaplasia or dysplasia. After modeling, the lotus leaf extract intervention groups were given high-dose (containing nuciferine of 20 mg/kg) and low-dose (containing nuciferine of 10 mg/kg) respectively by gavage, once a day. During the experiment, the rats' body weight and food intake were observed.

After 4 weeks of therapeutic administration, the food intake and body weight of the rats increased compared with the model-building stage, the hair color returned to bright, and the phenotypes such as recovery of activity ability returned to normal state. At the end of the administration, after the rats were sacrificed, the stomach tissue was quickly removed and prepared for pathological sectioning. The obtained gastric tissue was cut along the greater curvature of the stomach, washed with saline, blotted dry with filter paper, and fixed with formalin solution (10% formaldehyde solution). The sections were embedded, dehydrated by alcohol, and then made transparent with xylene. The sections were waxed and embed in paraffin. In the staining stage, the sections were soaked in xylene solution for 10 minutes each time for 3 times; then they were soaked in absolute ethanol and rinsed in tap water. Finally, the sections were stained with hematoxylin (to stain the nucleus) and eosin (to stain the cytoplasm), and were mounted with neutral gum. Finally, observation of the pathological changes of rat stomach tissue was made under light microscope.

TABLE 2
Experimental groupings of lotus leaf extract in
the treatment of chronic atrophic gastritis

| | | Number | | |
|---|---|---|---|---|
| | | in the | Total | |
| Grouping | Dosing content | group | number | label |
| Normal control | Normal saline of | 6 | 24 | A1-A6 |
| group | the same volume | | | |
| CAG model group | Normal saline of | 6 | | B1-B6 |
| | the same volume | | | |
| Lotus leaf extract | Dosage 2.5 g/kg | 6 | | C1-C6 |
| Low-dose group | (containing | | | |
| | 10 mg/kg | | | |
| | nuciferine) | | | |
| Lotus leaf extract | Dose 5 g/kg | 6 | | D1-D6 |
| High-dose group | (containing | | | |
| | 20 mg/kg | | | |
| | nuciferine) | | | |

### The Experimental Results:

1. Observing Pathological Sections of Stomach Tissue Under Light Microscope

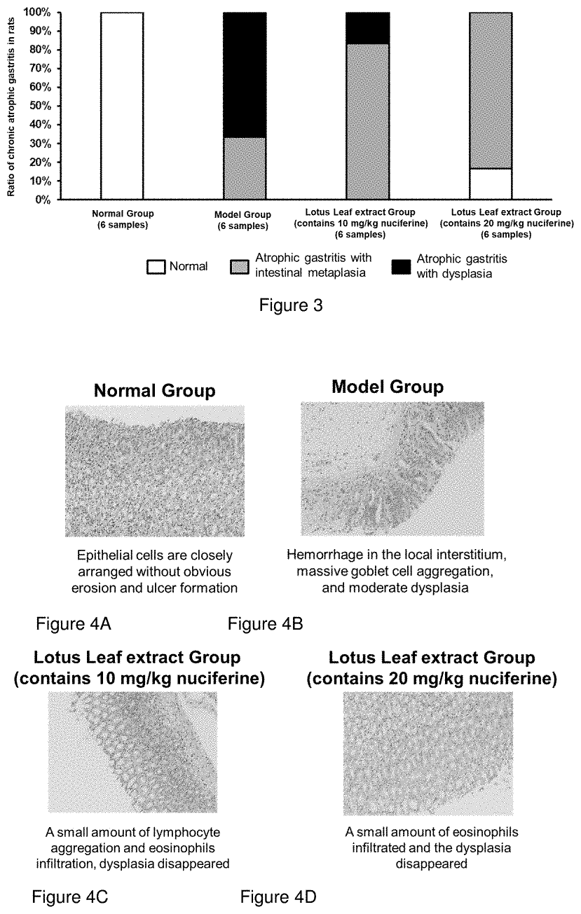

According to the interpretation results of pathological sections, compared with the rats the normal group, the rats of the model groups in the model-making stage developed moderate intestinal metaplasia or dysplasia pathology. The lotus leaf extract effectively treated chronic atrophic gastritis in the hyperplasia pathological state in rats, wherein the high-dose lotus leaf extract group had the best effect, with some rats had their stomach returned to a normal state, and the rest had their chronic atrophic gastritis weakened to a mild intestinal metaplasia pathological state. The results of pathological interpretation were statistically mapped, and the results obtained are shown in FIG. 3.

Observed under a light microscope, the gastric mucosa in the normal group had a smooth surface, clear texture, tightly arranged epithelial cells, and no obvious erosion and ulcer formation, as shown in FIG. 4A. In the chronic atrophic gastritis model group, focal interstitial hemorrhage was seen, a large number of cupped cells were gathered, and the glands were moderately dysplasia, as shown in FIG. 4B. In the low-dose lotus leaf extract group, the glandular dysplasia of some rats disappeared, and there was a small amount of lymphocyte aggregation and eosinophil infiltration, as shown in FIG. 4C. Compared with the model group, the high-dose lotus leaf extract group significantly reduced the pathological state, and some rats returned to normal state. Some rats had a little eosinophil infiltration, the glandular dysplasia disappeared, and the moderate intestinal metaplasia was reduced to mild intestinal metaplasia or disappeared, as shown in FIG. 4D.

### Embodiment 3: Nuciferine and Lotus Leaf Extract have Protective Effects on Gastric Mucosal Damage

The experimental: The experimental animals were from Beijing Sibeifu Biotechnology Co., Ltd., and 30 SD rats of 200±20 g specific pathogen free (SPF) grade, male, were chosen and randomly divided into 5 groups: normal control group, the gastric mucosal injury model group, the nuciferine low-dose group, the nuciferine high-dose group and the lotus leaf extract group, as shown in Table 3. The low-dose nuciferine group, the high-dose nuciferine group and the lotus leaf extract group were administered intragastrically for 14 days, once a day. The nuciferine was purchased from Chengdu Refines Biotechnology Co., Ltd., with a specification of 5 g and a purity of over 98%. The lotus leaf extract was purchased from Hebei Chenguang Biotechnology Group Handan Co., Ltd., with a specification of 1 kg and a content of nuciferine of more than 0.4%. 1 hour after the rats in the low-dose nuciferine group, the high-dose nuciferine group and the lotus leaf extract group were given continuous administration, the rats in the control group and the model group were strictly fasted for 24 hours (with water available), and medicine was also prohibited during this period. Rats in the model group, the low-dose nuciferine group, the high-dose nuciferine group, and the lotus leaf extract group were given 1.0 mL of absolute ethanol per rat by intragastric administration; 1 hour after the completion of the modeling, the rats were sacrificed by blood sampling from the abdominal aorta, and the stomach was taken by cutting the abdomen, was injected with 10% formaldehyde solution, and fixed for 20 minutes, then the stomach was cut along the greater curvature of the same and was washed to remove the stomach contents, and a general observation under a dissecting microscope was conducted and the length of injury (mm) was measured.

TABLE 3
Groups of experiments on protecting gastric mucosa

| | | Number | | |
|---|---|---|---|---|
| | | in the | Total | |
| Grouping | Dosing content | group | number | Label |
| Normal control | Normal saline of | 6 | 30 | A1-A6 |
| group | the same volume | | | |
| Gastric mucosal | Normal saline of | 6 | | B1-B6 |
| injury Model | the same volume | | | |
| group | | | | |
| Nuciferine low- | Dosage 20 mg/kg | 6 | | C1-C6 |
| dose group | | | | |
| Nuciferine high- | Dosage 40 mg/kg | 6 | | D1-D6 |
| dose group | | | | |
| Lotus Leaf Extract | Dosage 2.5 g/kg | 6 | | E1-E6 |
| Group | (containing | | | |
| | 10 mg/kg | | | |
| | nuciferine) | | | |

### The Experimental Results:

In the anatomy of the normal group and the model group, it was found that the rats in the model group had various degrees of gastric mucosal damage. In the nuciferine and lotus leaf extract groups, the gastric mucosal injury of rats was improved compared with the model group, and the rats' activity ability increased, indicating that nuciferine and lotus leaf extract can protect the gastric mucosa injury.

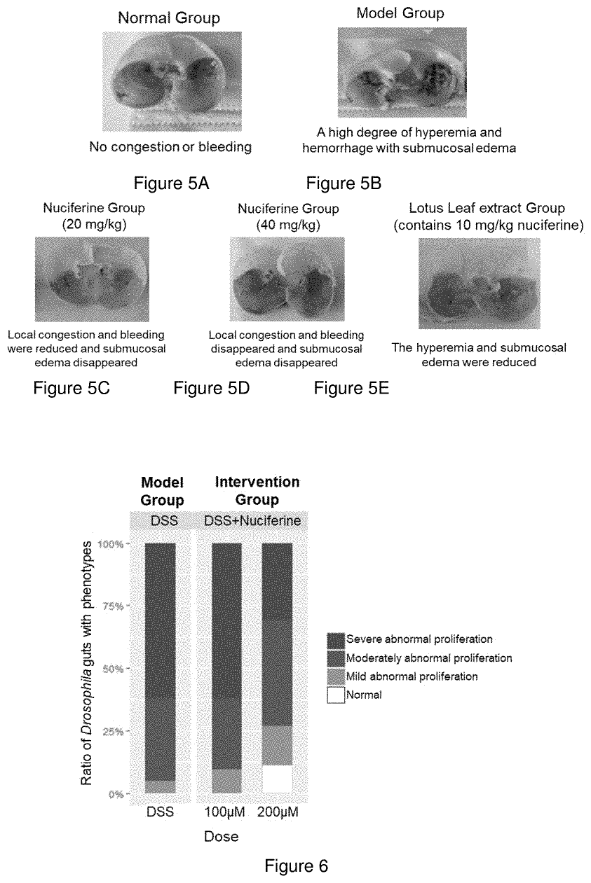

Results observed under the microscope after anatomy are shown in FIGS. 5A-5E. There was no congestion and bleeding in the normal group, as shown in FIG. 5A. The gastric mucosa of the model group was highly hyperemic and bleeding with submucosal edema, as shown in FIG. 5B. The local congestion and bleeding of the gastric mucosa in the low-dose nuciferine group were reduced, as shown in FIG. 5C. There was no congestion and bleeding in the gastric mucosa in the high-dose nuciferine group, as shown in FIG. 5D. In the lotus leaf extract group, gastric mucosal congestion and submucosal edema were alleviated, as shown in FIG. 5E.

### Embodiment 4: Nuciferine and Lotus Leaf Extract can Inhibit the Abnormal Proliferation of Gastrointestinal Stem Cells

The experimental: Dextran sulfate sodium (DSS) was used to induce abnormal proliferation of drosophila gastrointestinal stem cells to simulate the process of tumorigenesis. The experimental drosophila were esg-GFP purchased from Tsinghua Drosophila Center. GFP (green fluorescent protein driven by esg-Ga14) can specifically label intestinal stem cells and enteroblasts. A solution containing 20% sucrose, 3% DSS (average molecular weight 40 kDa) and 0.5% dimethyl sulfoxide (DMSO) was prepared, and the solution was mixed with the drosophila food, and the mixture was placed half-dry and was then used to feed the drosophila to perform modeling. During the modeling period, different groups were given nuciferine and lotus leaf extract. The nuciferine was purchased from Chengdu Refines Biotechnology Co., Ltd., with a specification of 5 g and a purity of over 98%. The lotus leaf extract was purchased from Hebei Chenguang Biotechnology Group Handan Co., Ltd., with a specification of 100 g and a nuciferine content of more than 0.4%. Sodium dextran sulfate was purchased from Beijing Zhongkekeao Biotechnology Co., Ltd. with a brand of mp and a specification of 50 g. The dimethyl sulfoxide was purchased from Amresco, USA, with a specification of 500 ml.

The Experiment Groups:

1) The Nuciferine Experiment Groups

Model control group: medium+0.5% DMSO+3% DSS

Low-dose nuciferine group: medium+0.5% DMSO+3% DSS+nuciferine 100 μM

High-dose nuciferine group: medium+0.5% DMSO+3% DSS+nuciferine 200 μM

2) The Lotus Leaf Extract Experiment Groups

Model control group: medium+0.5% DMSO+3% DSS

Lotus leaf extract low-dose group: medium+0.5% DMSO+3% DSS+lotus leaf extract (containing 100 μM lotus leaf extract)

Lotus leaf extract medium-dose group: medium+0.5% DMSO+3% DSS+lotus leaf extract (containing 200 μM lotus leaf extract)

Lotus leaf extract high-dose group: medium+0.5% DMSO+3% DSS+lotus leaf extract (containing 300 μM lotus leaf extract)

There were 3 parallel tubes per group. The virgin flies that hatched within one day were collected and reared in a normal medium at 25° C. for 3 days. The drosophila were transferred to the model group, the low-dose nuciferine group, the high-dose nuciferine group, the low-dose lotus leaf extract group, the lotus leaf extract medium-dose group, and the lotus leaf extract high dose group, with about 20 drosophila in each group, and were cultured for 3-5 days at 25° C. At the end of the experiment, the gastrointestinal tract was dissected and the changes in GFP were observed.

The Experimental Results:

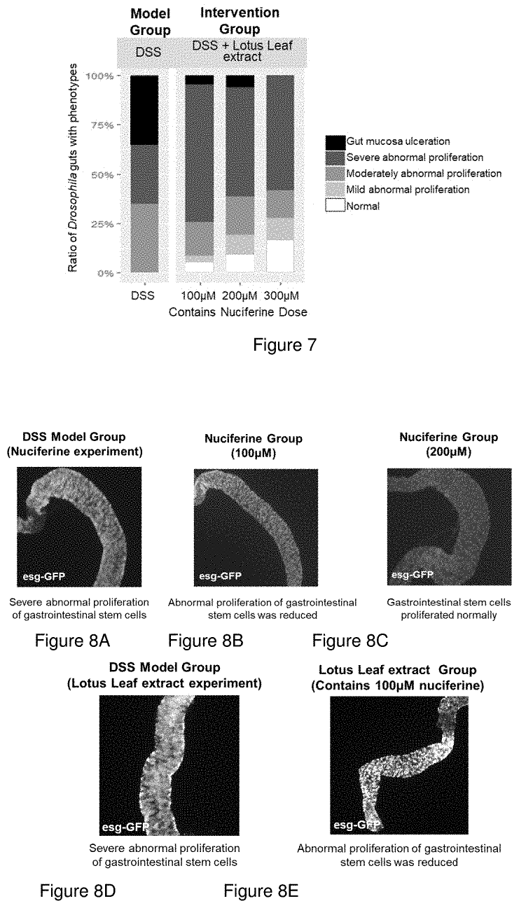

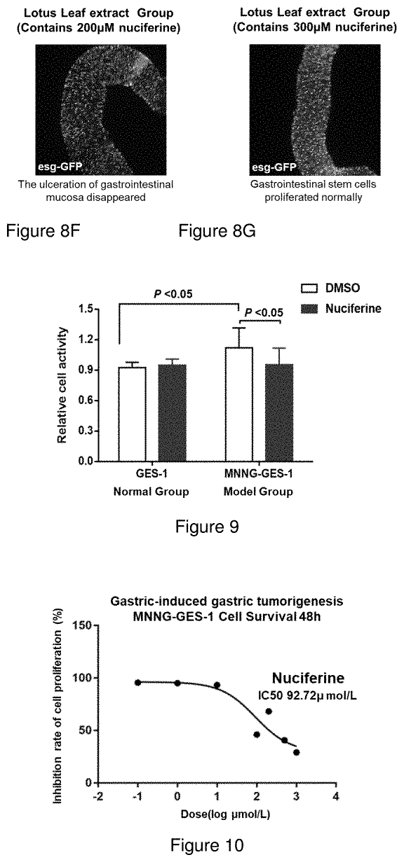

The statistical results under the microscope showed that the nuciferine and lotus leaf extract group effectively inhibited the abnormal proliferation of drosophila gastrointestinal stem cells and gastrointestinal mucosal ulceration. In the nuciferine experiment, the low-dose and high-dose nuciferine groups reduced the abnormal proliferation ratio of gastrointestinal stem cells, and the proliferation of some drosophila gastrointestinal stem cells returned to normal, as shown in FIG. 6. In the lotus leaf extract experiment, the lotus leaf extract reduced the abnormal proliferation of drosophila gastrointestinal stem cells to varying degrees as the dose increased; some drosophila gastrointestinal stem cells returned to normal proliferation, and the gastrointestinal mucosa ulcer disappeared, as shown in FIG. 7. The results of the dissection of the drosophila gastrointestinal tract under a microscope are shown in FIGS. 8A-8G. In the nuciferine experiment DSS model group drosophila gastrointestinal stem cells showed severe abnormal proliferation, as shown in FIG. 8A. The abnormal proliferation of drosophila gastrointestinal stem cells in the low-dose nuciferine group was reduced, as shown in FIG. 8B. In the high-dose nuciferine group, the proliferation of drosophila gastrointestinal stem cells returned to normal (see FIG. 8C). In the lotus leaf extract experiment DSS model group drosophila gastrointestinal mucosa was ulcerated and stem cells proliferated severely, as shown in FIG. 8D. In the low-dose lotus leaf extract group, the abnormal proliferation of drosophila gastrointestinal stem cells was reduced (see FIG. 8E). In the lotus leaf extract medium-dose group, the ulceration of the gastrointestinal mucosa of drosophila disappeared, and the abnormal proliferation of gastrointestinal stem cells was reduced, as shown in FIG. 8F. In the high-dose lotus leaf extract group, the ulceration of the gastrointestinal mucosa of fruit flies disappeared, and the proliferation of gastrointestinal stem cells returned to normal, as shown in FIG. 8G.

### Embodiment 5: Nuciferine has a Significant Inhibitory Effect on Gastritis Cancer Transformed Cell Model

The gastritis cancer transformation model used in this example formed by human gastric epithelial cells (GES-1) induced by chemical mutagens N-methyl N-nitronitrosoguanidine (MNNG) was as reported in the literature [21]. The nuciferine was purchased from Chengdu Refines Biotechnology Co., Ltd., with a specification of 1 g and a purity of over 98%. The MNNG was purchased from Beijing Baiyiyi Chuang Biotechnology Co., Ltd., with a brand Tixiai and a specification of 5 g. The GES-1 cells were purchased from Shanghai Xuran Biotechnology Co., Ltd. The phosphate buffered saline (PBS) was purchased from Amresco, USA, with a specification of 10 L. The dimethyl sulfoxide (DMSO) was purchased from Amresco, USA, with a specification of 500 ml. The fetal bovine serum (FBS) was purchased from Amresco, USA, with a specification of 100 ml. The 3-(4,5-Dimethylthiazole-2)-2,5-diphenyltetrazolium bromide (MTT) was purchased from Shanghai Biyuntian Biotechnology Co., Ltd., with a specification of 500 mg.

### The Experimental:

GES-1 cells was planted in a 96-well plate (8000 cells/well, leave a row of blank holes, discard the edge holes and add an equal volume of PBS to reduce evaporation), the old solution was aspirated the next day, and 10% FBS containing no double antibody medium was added, 2×10-5 MNNG was added to induce stimulation, culturing for 24 h in the dark, and discarding the culture medium containing MNNG. 7 concentration gradients of nuciferine (0.1 μM, 1 μM, 10 μM, 100 μM, 200 μM, 500 μM, 1000 μM) were set in the dosing group, with 3 replicate wells for each gradient; each well was added with the well-prepared nuciferine for 48 h, the old solution was aspirated, 0.5 mg/ml MTT-containing complete medium (containing 10% FBS, total volume 100 μl/well) was added, incubation was conducted at 37° C. for 4 h, the old solution was aspirated, 100 μl/DMSO was added, and after mixing with a horizontal shaker for 10 minutes absorbance was detected using an microplate reader.

### The Experimental Results:

Compared with the normal control group, the viability of GES-1 cells stimulated by MNNG was significantly enhanced, and nuciferine had the effect of significantly inhibiting the viability of MNNG-GES-1 cells (P<0.05), as shown in FIG. 9. Data processing of the experimental results showed that the half inhibitory concentration (IC50) of nuciferine on cells transformed from gastritis to gastric cancer was 92.72 μmol/L, as shown in Table 4 and FIG. 10, indicating that nuciferine has an inhibitory effect on cells transformed from gastritis to gastric cancer.

### Embodiment 6: Lotus Leaf Extract Improves Symptoms and Objective Indicators of Patients with Chronic Atrophic Gastritis or Gastric Precancerous Lesions

The Experiment:

In this experiment, patients with chronic atrophic gastritis with intestinal metaplasia or dysplasia were treated with lotus leaf extract, and the curative effect was evaluated by investigating the patient's related blood indicators and improvement of clinical symptoms. The lotus leaf extract was provided by Hebei Chenguang Biotechnology Group Handan Co., Ltd. According to the new Sydney criteria, patients who were pathologically diagnosed as chronic atrophic gastritis or with intestinal metaplasia or dysplasia were included. After filling out the informed consent form, the subjects drank 1 bag (10 g/bag) of lotus leaf extract every day, and gave feedback on the improvement of chronic atrophic gastritis symptoms once a week. After drinking for 2 months, they went to the hospital for review and blood-related indicators (serum Gastrin 17). Serum gastrin 17 plays a role in promoting the occurrence and development of gastric cancer. When the level of serum gastrin 17 increases, it indicates the risk of gastric cancer. Serum gastrin 17 has a promoting effect in the occurrence and development of gastric cancer. When the level of serum gastrin 17 increases, it indicates the risk of gastric cancer [25]. In this experiment, there were 10 patients who were tested for symptom statistics. The overall situation of the tested patients was: 3 males, 7 females; 2 people aged 45-50 years old, 1 person 50-55 years old, 4 people 60-65 years old, and 3 people 65-70 years old; before the test 3 patients had been diagnosed to have chronic atrophic gastritis, 3 patients had been diagnosed to have chronic atrophic gastritis with intestinal metaplasia, and 4 patients had been diagnosed to have chronic atrophic gastritis with intestinal metaplasia and dysplasia. Among them, 2 patients had been tested for blood index, one of whom was a 60 years old female who had been diagnosed as chronic atrophic gastritis with moderate intestinal metaplasia and mild dysplasia before the test and the other one of whom was a 52 years old female who had been diagnosed as chronic atrophic gastritis before the test.

The Experimental Results:

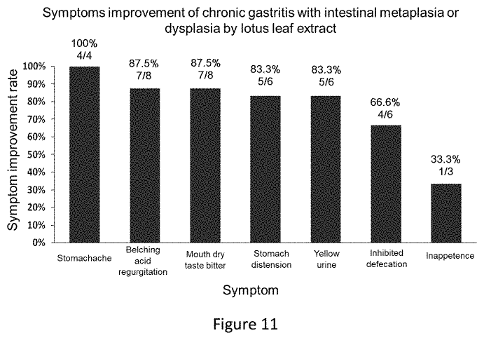

As shown in Table 5, before drinking lotus leaf extract, blood test index serum gastrin of patient 1 had been high at 25.84 pmol/L, and the index returned to normal range after drinking lotus leaf extract for 2 months. Patient 2 had had blood test index serum gastrin of a high value of 17 pmol/L, and the index returned to the normal range after drinking lotus leaf extract for 2 months (as shown in Table 5). As shown in FIG. 11, through statistics of the symptoms of the tested patients before and after drinking lotus leaf extract, it was found that lotus leaf extract was capable of relieving the symptoms of chronic atrophic gastritis; for example, the improvement rate of stomach pain was 100%, the improvement rate of belching acid was 87.5%, and the improvement rate of gastric bloating was 83.3%. The above experimental results show that lotus leaf extract can effectively improve the symptoms and objective indicators of patients with chronic atrophic gastritis with intestinal metaplasia or dysplasia.

TABLE 5
Effect of lotus leaf extract on blood index improvement in patients
with atrophic gastritis or gastric precancerous lesions

| | Serum gastrin 17 (pmol/L) |
|---|---|
| Patient 1 (before drinking) | 25.84 (too high) |
| Patient 1 (after drinking) | 3.71 |
| Patient 2 (before drinking) | 16.25 (too high) |
| Patient 2 (after drinking) | 1.99 |

The experimental results of the above embodiments comprehensively show that nuciferine and lotus leaf extract can effectively treat chronic atrophic gastritis and gastric precancerous lesions, prevent and/or block the transformation of gastritis to gastric cancer; moreover, lotus leaf extract and lotus leaf extract are safe and can protect the gastric mucosa from damage. Thus, it can be seen that nuciferine and lotus leaf extract can be used as drugs, health products and/or food for effective treating chronic atrophic gastritis, treating gastric precancerous lesions and/or blocking the occurrence of transformation of gastritis to gastric cancer; and nuciferine and lotus leaf extract can be used as an effective component of medicines, health products and/or foods that effectively treat chronic atrophic gastritis, treat gastric precancerous lesions and/or block the occurrence of transformation of gastritis to gastric cancer.

### REFERENCES

1. Crusz S M, Balkwill F R. Inflammation and cancer: advances and new agents. Nature Reviews Clinical Oncology, 2015, 12(10):584-96.
2. Grinberg-Bleyer Y, Ghosh S. A Novel Link between Inflammation and Cancer. Cancer Cell, 2016, 30(6):829-830.
3. Anand P, Kunnumakara A B, Sundaram C, et al. Cancer is a Preventable Disease that Requires Major Lifestyle Changes. Pharmaceutical Research, 2008, 25(9):2097-2116.
4. Ohata H, Kitauchi S, Yoshimura N, et al. Progression of chronic atrophic gastritis associated with Helicobacter pylori infection increases risk of gastric cancer. International journal of cancer, 2004, 109(1): 138-143.
5. Uemura N, Okamoto S, Yamamoto S, et al. Helicobacter pylori infection and the development of gastric cancer. New England Journal of Medicine, 2001, 345(11): 784-789.
6. Chen W, Zheng R, Baade P D, et al. Cancer statistics in China, 2015. CA: a Cancer Journal for Clinicians, 2016, 66(2): 115-132.
7. Zuo Tingting, Zheng Rongshou, Zeng Hongmei, et al. The Epidemiology of Gastric Cancer in China. Chinese Journal of Oncology, 2017, 44(1): 52-58.
8. Grivennikov S I, Greten F R, Karin M. Immunity, Inflammation, and Cancer. Cell, 2010, 140(6):883-899.
9. Ziech D, Franco R, Pappa A, et al. Reactive Oxygen Species (ROS)-Induced genetic and epigenetic alterations in human carcinogenesis. Mutation Research/fundamental & Molecular Mechanisms of Mutagenesis, 2011, 711(1-2):167-73.
10. Denicola G M, Karreth F A, Humpton T J, et al. Oncogene-induced Nrf2 transcription promotes ROS detoxification and tumorigenesis. Nature, 2011, 475(7354):106-9.
11. Benamouzig R, Uzzan B. Aspirin to prevent colorectal cancer: time to act?. Lancet, 2010, 376(9754):1713-4.
12. Zhu Lanzhen, Li Wei. Study on the effect of lotus leaf total alkaloid extract on blood lipid regulation in hyperlipidemic rats. Heilongjiang Medicine, 2010, 23(3):363-364.
13. Guo F C, Yang X, Li X X, et al. Nuciferine prevents Hepatic Steatosis and injury induced by a high-fat diet in Hamsters. PLoS One, 2013, 8(5):1-10.
14. Kuang Jun, Wang Wei. The effect of nuciferine on atherosclerotic vascular inflammation and matrix metalloproteinases in mice. Journal of Clinical Cardiovascular Disease, 2015, 31(1): 97-100.
15. Li M Y, Xu Z T. The inhibition of dentifrice containing the lotus leaf-derived inhibitor on periodontitis-related bacteria in vitro. Int Dent, 2007, 57(10):303-306.
16. Liu W, Yi D D, Guo J L, et al. Nuciferine, extracted from Nelumbo nucifera Gaertn, inhibits tumor-promoting effect of nicotine involving Wnt/β-catenin signaling in non-small cell lung cancer. Journal of Ethnopharmacology, 2015, 165(15): 83-93.
17. Tao Bo, Shuai Jingxian, Wu Fenglian. The effect of lotus leaf decoction on blood lipids and hemorheology in hyperlipidemia rats. Journal of Chinese Medicine, 2000, 28(6): 55-56.
18. Kong Wenqi, Li Yanwei. Research progress on active chemical constituents and pharmacology of lotus leaf. Research and Information on Chinese Medicine, 2005(6): 22-24.
19. Li Shao, Li Huiying Chinese Patent CN201310455974.4, 2018.1.8.
20. Si J, Zhou W, Wu J, et al. Establishment of an animal model of chronic atrophic gastritis and a study on the factors inducing atrophy. Chinese Medical Journal, 2001, 114(12):1323-1325.
21. Chen S, Zhong J, Zhou Q, et al. The Regenerating Gene Iα Is Overexpressed in Atrophic Gastritis Rats with Hypergastrinemia. Gastroenterology Research and Practice, 2011, 2011(5):403956.
22. Chauhan A K, Kang S C. Therapeutic potential and mechanism of thymol action against ethanol-induced gastric mucosal injury in rat model. 2015, 49(7):739-45.
23. Alla A, Jiang J, Tony Y. LP. Tissue Damage-Induced Intestinal Stem Cell Division in Drosophila. Cell Stem Cell, 2009, 4:49-61.
24. Yang Q, Jie Z, Ye S, et al. Genetic variations in miR-27a gene decrease mature miR-27a level and reduce gastric cancer susceptibility. Oncogene, 2014, 33(2):193.
25. Sun L, Tu H, Liu J, et al. A comprehensive evaluation of fasting serum gastrin-17 as a predictor of diseased stomach in Chinese population. Scand J Gastroenterol, 2014, 49(10): 1164-1172.

1. Use of a substance for preparing a product to treat chronic atrophic gastritis, wherein said product is one selected from:
medicine,
health care products, and
food,
and the substance is one selected from:
nuciferine,
nuciferine derivatives,
lotus leaf extracts, and
a mixture containing at least two selected from the group consisting of nuciferine, nuciferine derivatives, and lotus leaf extracts.

2. The use according to claim 1, wherein the nuciferine belongs to apophine-type alkaloid compound with the molecular formula of C19H21NO2 and the molecular weight of 295.38 g/mol and the structural formula:

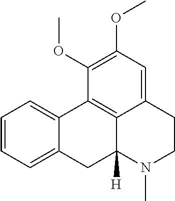 [SMILES: COc1cc2c3c(c1OC)-c1ccccc1C[C@H]3N(C)CC2]

3. Use of a substance for preparing a product for blocking the occurrence of transformation of gastritis to gastric cancer, wherein said product is one selected from:
medicine,
health care products, and
food,
and the substance is one selected from:
nuciferine,
nuciferine derivatives,
lotus leaf extracts, and
a mixture containing at least two selected from the group consisting of nuciferine, nuciferine derivatives, and lotus leaf extracts.

4. The use according to claim 3, wherein the nuciferine belongs to apophine-type alkaloid compound with the molecular formula of C19H21NO2 and the molecular weight of 295.38 g/mol and the structural formula:

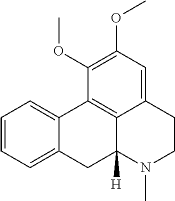 [SMILES: COc1cc2c3c(c1OC)-c1ccccc1C[C@H]3N(C)CC2]

5. A product containing as an active ingredient one selected from:
nuciferine,
nuciferine derivatives,
lotus leaf extracts,
a mixture containing at least two selected from the group consisting of nuciferine, nuciferine derivatives, and lotus leaf extracts, and
other accessories,
wherein said product is one selected from medicine, health care products, and food.

6. The product according to claim 5, wherein the product is used for at least one of treating chronic atrophic gastritis, protecting gastric mucosa, blocking transformation of gastritis to gastric cancer, treating gastric precancerous lesions, and preventing the occurrence of gastric cancer.

7. The product according to any one of claims 5-6, wherein the dosage form of the medicines, health products or foods is one selected from tablets, capsules, pills, injections, sustained-release agents, controlled-release agents, powders, beverages.

8. The use of the substance for preparing the product to treat the chronic atrophic gastritis, according to claim 1, wherein the product is further used for at least one of protection of gastric mucosa, block transformation of gastritis to gastric cancer, treatment of gastric precancerous lesions, and prevention of gastric cancer.

9. The use of the substance for preparing the product to treat the chronic atrophic gastritis, according to claim 2, wherein the mixture is further used for at least one of protection of gastric mucosa, prevention of transformation of gastritis to gastric cancer, treatment of gastric precancerous lesions, and prevention of gastric cancer.

10. The use of the substance for preparing the product for blocking the occurrence of transformation of gastritis to gastric cancer according to claim 3, wherein the mixture is further used for treating chronic atrophic gastritis, protecting gastric mucosa, treating gastric precancerous lesions, and preventing gastric cancer.

11. The use of the substance for preparing the product for blocking the occurrence of transformation of gastritis to gastric cancer according to claim 4, wherein the mixture is further used for treating chronic atrophic gastritis, protecting gastric mucosa, treating gastric precancerous lesions, and preventing gastric cancer.
